(19) United States
(12) Patent Application Publication
(10) Pub. No.: US 2013/0085341 A1
(43) Pub. Date: Apr. 4, 2013

(54) METHODS AND DEVICES FOR MANIPULATING TISSUE IN VIVO

(71) Applicants: Rudolph H. Nobis, Mason, OH (US); Robert M. Trusty, Cincinnati, OH (US); James T. Spivey, Cincinnati, OH (US); Christopher J. Hess, Cincinnati, OH (US); Per O. Park, Gothenburg (SE); Omar J. Vakharia, Cincinnati, OH (US)

(72) Inventors: Rudolph H. Nobis, Mason, OH (US); Robert M. Trusty, Cincinnati, OH (US); James T. Spivey, Cincinnati, OH (US); Christopher J. Hess, Cincinnati, OH (US); Per O. Park, Gothenburg (SE); Omar J. Vakharia, Cincinnati, OH (US)

(21) Appl. No.: 13/249,896

(22) Filed: Sep. 30, 2011

(51) Int. Cl. A61B 1/32; A61B 17/34

(52) U.S. Cl. 600/213; 606/185

(57) ABSTRACT

Methods and devices are provided for manipulating tissue in a body cavity, i.e., in vivo. In general, the methods and devices allow an end effector to engage tissue, be detached from a shaft used to control the end effector to engage the tissue, and then be moved to another location in the body cavity by a separate component, such as an external control unit. In some embodiments the end effector can include one or more deployable hooks that allow the end effector to remain at a surgical site independent of any shafts or external control units. Still further, in other embodiments the end effector can include a transverse bore to allow a second shaft to connect to the end effector to manipulate the tissue. Additional exemplary devices, kits, and methods related to manipulating tissue in vivo are also provided.

### FIELD

The present disclosure relates to methods and devices for manipulating and moving tissue at a surgical location, and more particularly to methods and devices for moving tissue engaged by end effectors that are detached from their operable instruments.

### BACKGROUND

Minimally invasive surgical techniques such as endoscopies and laparoscopies are often preferred over traditional open surgeries because the recovery time, pain, and surgery-related complications are typically less with minimally invasive surgical techniques. In many laparoscopic procedures, the abdominal cavity is insufflated with carbon dioxide gas. The abdominal wall is pierced and a cannula or trocar is inserted into the abdominal cavity. Surgeons can then perform a variety of surgical procedures while minimizing patient trauma.

Many procedures require a variety of different functions be performed at the surgical site. These functions can be performed by different instruments, each being tailored to perform a particular function. Alternatively, a single instrument having interchangeable and replaceable end effectors can be used, with each end effector being tailored to perform a particular function. Often, multiple instruments or end effectors are disposed or used at a surgical site at the same time, and thus multiple surgical access ports are used to accommodate the instruments. In addition to an end effector designed to perform a particular function at the surgical site, the instruments can include a handle to operate the end effector from outside of a subject and a shaft connecting the handle and end effector. The diameter of the end effector is usually substantially larger than the shaft, and thus techniques have been developed that allow end effectors to be introduced through a single, larger port, while shafts are introduced to the surgical site through smaller incisions or ports.

One issue that arises from using multiple instruments at the same time is the congestion that results. The end effectors and shafts of the instruments are obstacles the surgeon must contend with while trying to perform delicate procedures. Another issue faced by surgeons is the number of available ports for use during a surgical procedure. As currently designed, each instrument being used at the surgical site typically requires its own port for operating the end effector associated with the instrument. Thus, when multiple instruments are disposed at a surgical site at the same time, multiple ports, and thus multiple incisions, are formed. The resulting trauma from the formation of ports and incisions may heal over time, but can be painful to the patient immediately after surgery and may also lead to undesirable scars and/or cosmetic procedures to minimize the effects of the trauma.

Accordingly, it is desirable to develop methods and devices that assist in minimizing the congestion that results from performing surgical procedures using multiple instruments in a small space. It is also desirable to develop methods and devices that minimize the number of ports being occupied by instruments located at a surgical site.

### SUMMARY

Methods and devices are generally provided for manipulating tissue in a subject's body, i.e., in vivo. In one embodiment a method for manipulating body tissue includes grasping tissue in a body cavity with an end effector attached to a shaft, detaching the end effector from the shaft within the body cavity, and then manipulating the end effector to move the grasped tissue that is secured to the end effector to a second location within the body cavity. The end effector is in fact removably and replaceably attached to the first shaft, which itself extends through a patient's tissue and into the body cavity to operate the end effector when the end effector is attached to the shaft.

In one embodiment, prior to even grasping the tissue, the first shaft can be inserted through a first port and the end effector can be inserted through a second port. After the end effector grasps the tissue, the end effector can be locked to the grasped tissue. The step of manipulating the end effector can be effected, for example, by using an external control unit disposed outside of the body cavity. The external control unit can include a magnetic material that is complementary to a material of the end effector. As various movements are imparted on the external control unit, the complementary end effector can move within the body cavity.

Alternatively, the step of manipulating the end effector can be effected by inserting a second shaft into the body cavity, coupling the second shaft to the end effector at a location that is different from the point at which the first shaft is coupled to the end effector, and manipulating the second shaft to move the grasped tissue to a second location. The first shaft can be inserted through a first port, the end effector through a second port, and the second shaft through a third port. Alternatively, the first shaft can be inserted through a first port and both the end effector and the second shaft can be inserted through a second port. The insertion of the second shaft can occur while the first shaft is coupled to the end effector. Further, the first shaft can be mated within a first bore of the end effector and the second shaft can be mated within a second bore of the end effector, with the second bore being substantially perpendicular to the first bore.

In one embodiment the method can include attaching the end effector in which tissue is grasped to other tissue at the surgical site to maintain the end effector at a desired location of the surgical site. For example, the end effector can be attached at the second location to maintain the end effector at the second location. This can be achieved, for instance, by hooking the end effector to the other tissue using a hook included as part of the end effector.

In one embodiment of a device for manipulating tissue, the device includes an end effector having one or more selectively deployable hooks disposed within the end effector and configured to selectively engage tissue to maintain the end effector at a desired location. A tissue grasping component can be formed at the distal end of the end effector and can be configured to engage tissue. The tissue grasping component can be configured to engage a first tissue prior to selectively engaging the hooks into additional tissue. Meanwhile, the end effector can be configured to be selectively moved from a first location to a second location by a movement control component.

In one embodiment a first bore can be disposed in the proximal end of the end effector and can extend along a longitudinal axis of the end effector. The bore can be configured to mate with a shaft, such as the shaft of an instrument, that is effective to operate the end effector to selectively grasp and release tissue. The bore and the shaft can mate by way of a threaded connection. Further, the tissue grasping component can include a pair of movable opposed jaws. In one embodiment a second bore can extend through at least part of each of the jaws and can be oriented substantially perpendicular to the longitudinal axis of the end effector. A second shaft can be mated to at least a portion of the second bore by way of a threaded connection.

In another aspect, a kit for manipulating tissue includes one or more end effectors and an external control unit configured to move the end effector from a first location to a second location. The end effectors can be capable of having a number of different functionalities, including but not limited to grasping tissue. The kit can also include a threaded shaft. The shaft can include a tissue penetrating distal tip that is configured to be inserted percutaneously through tissue. In some embodiments, the end effector can include a first bore at its proximal end that is oriented along a longitudinal axis of the end effector and a second bore that is distal to the first bore and oriented substantially perpendicular to the first bore. The end effector can be inserted into a body cavity independent of the threaded shaft and can then be selectively attached to and detached from the threaded shaft in vivo. Optionally, the kit can include an end effector that has one or more selectively deployable hooks disposed therein, a trocar element, and/or other components used in conjunction with laparoscopic or endoscopic procedures.

### BRIEF DESCRIPTION OF DRAWINGS

This invention will be more fully understood from the following detailed description taken in conjunction with the accompanying drawings, in which:

FIG. 1 is a schematic view of one exemplary embodiment of a single port surgical procedure with percutaneous device assistance;

FIG. 2A is a schematic view of one exemplary embodiment of a step in a surgical procedure that includes a detachable end effector, illustrating the end effector attached to a shaft to grasp tissue;

FIG. 2B is a schematic view of the surgical procedure of FIG. 2A, illustrating the end effector detached from the shaft while grasping tissue;

FIG. 2C is a schematic view of the surgical procedure of FIG. 2B, illustrating an external control unit controlling the detached end effector to move it from a first position to a second position;

FIG. 2D is a schematic view of the surgical procedure of FIG. 2C, illustrating the external control unit controlling the end effector to move the tissue from a first location to a second location;

FIG. 3 is a schematic view of another exemplary embodiment of a surgical procedure including multiple end effectors;

FIG. 4 is a schematic view of yet another exemplary embodiment of a surgical procedure including a detached end effector with a suture attached thereto;

FIG. 5 is a side view of one exemplary embodiment of an instrument coupled to an end effector;

FIG. 6 is a side view of the end effector detached from a shaft of the instrument of FIG. 5;

FIG. 7 is an isometric side view of a distal end of the shaft of the instrument of FIG. 5 in a locked position;

FIG. 8 is an isometric side view of the distal end of the shaft of FIG. 7 in an unlocked position;

FIG. 9 is a partially-transparent side view of the end effector of FIG. 5;

FIG. 10 is a partially-transparent side view of the shaft of FIG. 5, including an obturator;

FIG. 11 is a partially-transparent, isometric view of a proximal portion of the end effector of FIG. 9;

FIG. 12 is a partially-transparent, cross-sectional view of the end effector and shaft of FIG. 5 in an assembled, unlocked position;

FIG. 13 is a partially-transparent, cross-sectional view of the end effector and shaft of FIG. 12 in an assembled, locked position;

FIG. 14A is a partially-transparent, detail view of the end effector and shaft, including an obturator, of FIG. 13;

FIG. 14B is a partially-transparent, isometric view of the FIG. 14A;

FIG. 15 is a side view of a handle of the instrument of FIG. 5;

FIG. 16 is an isometric view of one exemplary embodiment of an end effector attached to a shaft of an instrument, the end effector being in a closed position;

FIG. 17 is an isometric view of the end effector and shaft of FIG. 16, the end effector being in an open position;

FIG. 18 is an isometric view of the end effector and shaft of FIG. 16, the end effector including a deployable hook;

FIG. 19 is an isometric view of the end effector and shaft of FIG. 18, the end effector being detached from the shaft; and

FIG. 20 is a schematic view another exemplary embodiment of an end effector and a shaft of an instrument, also including a second shaft.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The methods and devices disclosed herein are generally directed to a surgical method for moving or manipulating tissue in vivo. In one exemplary embodiment a device or instrument for performing a surgical procedure includes a detachable end effector. The end effector can be initially controlled by the instrument to perform a certain task, for instance grasping tissue. After the task is completed, the end effector can be detached from the instrument and moved and manipulated at the surgical site using mechanisms other than the instrument that initially controlled it, such as additional shafts or an external control unit capable of communicating with the end effector to control movement of the end effector without a shaft. External control units and end effectors can communicate, for example, by having complementary magnets associated therewith. To further improve surgical procedures, the end effector can also include one or more deployable hooks, which can be used to engage adjacent tissue to temporarily maintain a location of the end effector at a surgical site while no instruments or other mechanisms are coupled to or associated with the end effector.

By allowing an end effector to be movable separate from an instrument that initially controlled it, the number of incisions used during the course of a surgical procedure can be minimized, as can the number of instruments and/or shafts disposed in the subject's body during the course of the procedure. For instance, by using an external control unit to manipulate the end effector, a separate incision and instrument shaft may be avoided, along with any need to use additional instruments to help control and move the end effector. Limiting the number of instruments disposed at the surgical site eases movement and manipulation of the end effectors, and tissue associated therewith, because there are fewer obstacles at the site. Further, if multiple end effectors are to be disposed at the surgical site at the same time, and left in place independent of the instrument used to insert it, each end effector does not require its own port to be introduced to the surgical site. The end effectors can be held in place, such as by an external control unit or by a mechanism to secure them in a desired location, while other end effectors are introduced through the same port as the first end effector.

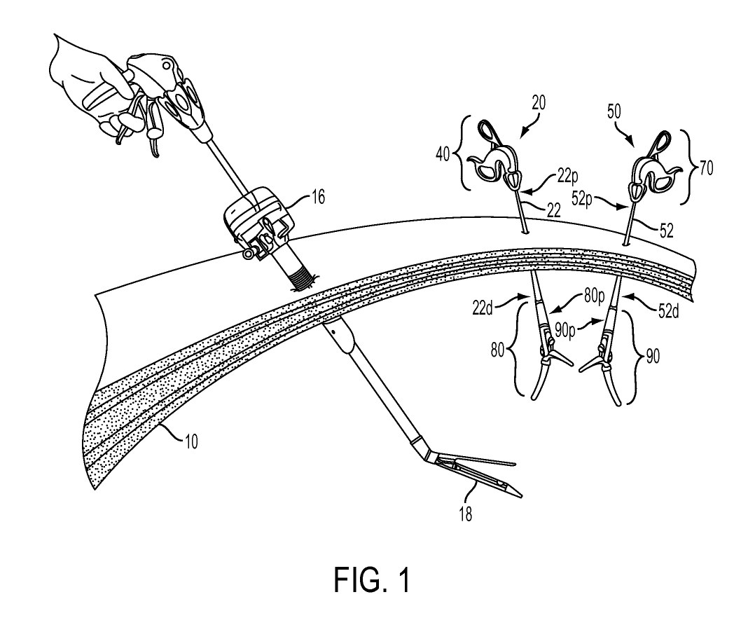

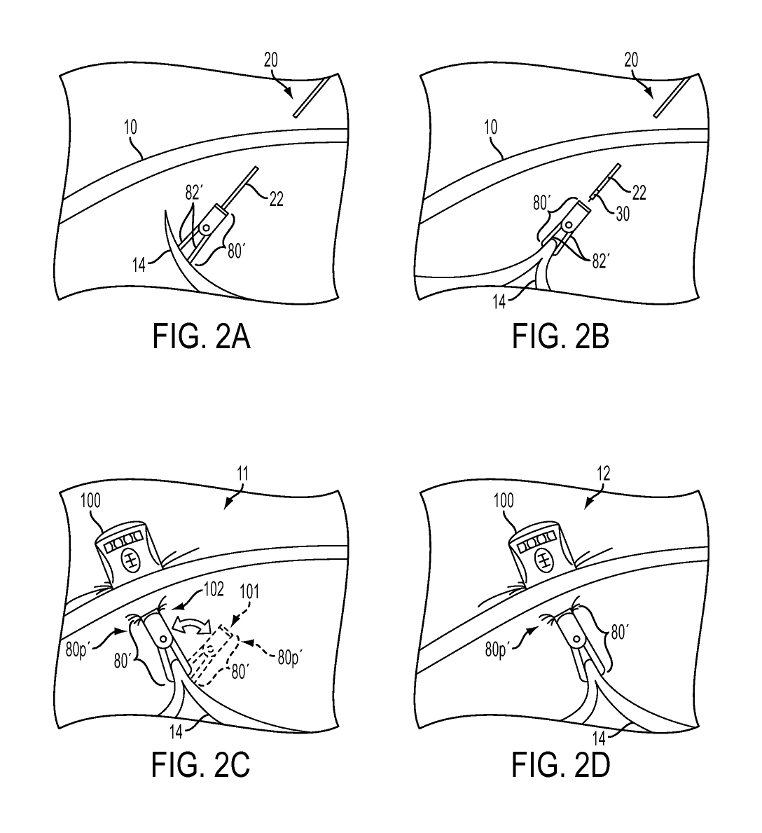

FIGS. 1-2D illustrate one exemplary embodiment of a method for manipulating tissue. As shown in FIG. 1, a single access port 16 is placed in an abdominal wall 10. The port location can be in the patient's umbilicus, however, other locations can also be used. Further, one or more percutaneous instruments 20, 50 can be used as part of the procedure. Each instrument 20, 50 can include an elongate shaft 22, 52 configured to receive one or more end effectors 80, 90 at a distal end 22d, 52d thereof, and handles 40, 70 configured to be coupled to a proximal end 22p, 52p of the shaft 22, 52 such that the handles 40, 70 can operate the end effectors 80, 90. As shown, the distal ends 22d, 52d of the shafts 22, 52 can pass through the abdominal wall 10 independent of a trocar or other access port, and the resilient abdominal wall 10 seals directly against the shafts 22, 52 to maintain pneumoperitoneum during a procedure. For example, the instruments 20, 50 can include an obturator (not shown) disposed within the shafts 22, 52, which can be selectively exposed to puncture tissue, or the distal ends 22d, 52d can include tissue-penetrating tips. The shafts 22, 52 can have very small diameters in comparison to traditional laparoscopic instruments leaving little, if any, postoperative scarring or patient discomfort.

In the illustrated embodiment, the end effectors 80, 90 are shown attached to the instruments 20, 50 within an abdominal cavity. The end effectors 80, 90 can be attached to the instruments 20, 50 in a number of different ways either outside or inside of a subject's body. In an exemplary embodiment, the end effectors 80, 90 can be configured to be attached to the elongate shafts 22, 52 within a patient's body or in vivo, e.g., within a body cavity such as the abdominal cavity, and to be removed and replaced within the patient's body. In this way, the elongate shafts 22, 52 can be introduced into the patient's body once and used in a variety of different ways, e.g., with a variety of different end effectors, without being removed from the patient's body, which can save time during a surgical procedure, reduce tissue trauma, and/or minimize the chance of infection.

As shown, to help facilitate mating the end effectors 80, 90 to the substantially cylindrical elongate shafts 22, 52 of the instruments 20, 50, a proximal portion 80p, 90p of the end effectors 80, 90 can have a substantially cylindrical shape. Proximal portions 80p, 90p can further be configured to removably and replaceably couple the end effectors 80, 90 to the shafts 22, 52 using an attachment mechanism 30 (shown in FIG. 2B), e.g., interference fit, snap fit, threads, etc.

A variety of different end effectors 80, 90 can be attached and detached from the instruments 20, 50. In the illustrated embodiment the end effectors 80, 90 are Maryland dissectors, and as also shown, another example of an end effector is a surgical stapler 18. As known in the art, a loader (not shown) can be passed through the access port 16 to facilitate attaching and detaching the end effectors 80, 90 to/from the shafts 22, 52 so that the size of the incisions associated with the percutaneous instruments 20, 50 can remain small. Exemplary embodiments of methods and devices for removably and replaceably coupling an end effector to an elongate shaft, including by use of a loader, are described in further detail in U.S. Patent Application Publication No. 2011/0087265, entitled “Laparoscopic Instrument with Attachable End Effector,” filed Oct. 9, 2009, U.S. Patent Application Publication No. 2011/0087266, entitled “Loader for Exchanging End Effectors In Vivo,” filed Oct. 9, 2009, U.S. Patent Application Publication No. 2011/0087267, entitled “Method for Exchanging End Effectors In Vivo,” filed Oct. 9, 2009, U.S. Patent Application Publication No. 2010/0063538, entitled “Surgical Grasping Device,” filed Sep. 9, 2008, U.S. patent application Ser. No. 12/873,845, entitled “Improved Minimally Invasive Surgery,” filed Sep. 1, 2010, U.S. Pat. No. 5,441,059, entitled “Method of Minimally Invasive Surgery,” issued on Aug. 15, 1995, and U.S. Pat. No. 5,352,219, entitled “Modular Tools for Laparoscopic Surgery,” issued on Oct. 4, 1994, which are hereby incorporated by reference in their entireties.

In use, a variety of end effectors can be selectively attached and detached from the instruments 20, 50 and subsequently manipulated and moved independent of the instruments 20, 50. FIGS. 2A-2D illustrate one example of a procedure using a single end effector 80′.

As shown in FIG. 2A, the instrument 20 is disposed in the abdominal wall 10 and has attached to its shaft 22 an end effector 80′ having jaws 82′ configured to grasp tissue within a body cavity. The instrument 20 is operated by the handle 40 (not shown) to cause the end effector 80′ to grasp tissue 14 and subsequently lock its jaws 82′ so that the tissue 14 remains grasped therein even after the instrument 20 is disassociated with the end effector 80° .

As shown in FIG. 2B, after the function of the end effector 80′ is performed, the instrument 20 can be detached from the end effector 80′ and removed from the surgical site. In the illustrated embodiment the attachment mechanism 30 is configured to be snap fit with the end effector 80′ such that operation of a control mechanism on the handle 40 (not shown) of the instrument disengages the attachment mechanism 30 from the end effector 80′.

As shown in FIG. 2C, a movement control component or external control unit 100 can be located proximate to the abdominal wall 10 to allow the external control unit 100 to communicate with the end effector 80′ transcutaneously. In the illustrated embodiment the external control unit 100 includes a magnetic element that is complementary to a magnetic element included as part of a proximal end 80p′ of the end effector 80′, thereby causing the proximal end 80p′ to move from a first position 101 to a second position 102 when the external control unit 100 is close enough to the end effector 80′ to cause a magnetic force to exist therebetween. The external control unit 100 can then be moved in any desired manner, including laterally, longitudinally, and rotationally, to impart corresponding movement to the end effector 80′. As shown between FIGS. 2C and 2D, the external control unit 100, and thus the end effector 80′ and associated tissue 14, are moved laterally across the abdominal cavity from a first position 11 to a second position 12.

Once the tissue 14 reaches it desired location, any further surgical procedures can be performed. For example, one or more instruments, such as the surgical stapler 18 illustrated in FIG. 1, can be introduced to the surgical site to act on the tissue 14 to set the tissue 14 at its new location. Further, the instrument 20 can be reintroduced to the surgical site to reattach to the end effector 80′. The instrument 20 can then be operated to unlock the end effector 80′ from the tissue 14. A person skilled in the art can appreciate a number of other procedures that can be performed following movement of the tissue 14 from the first position 11 to the second position 12.

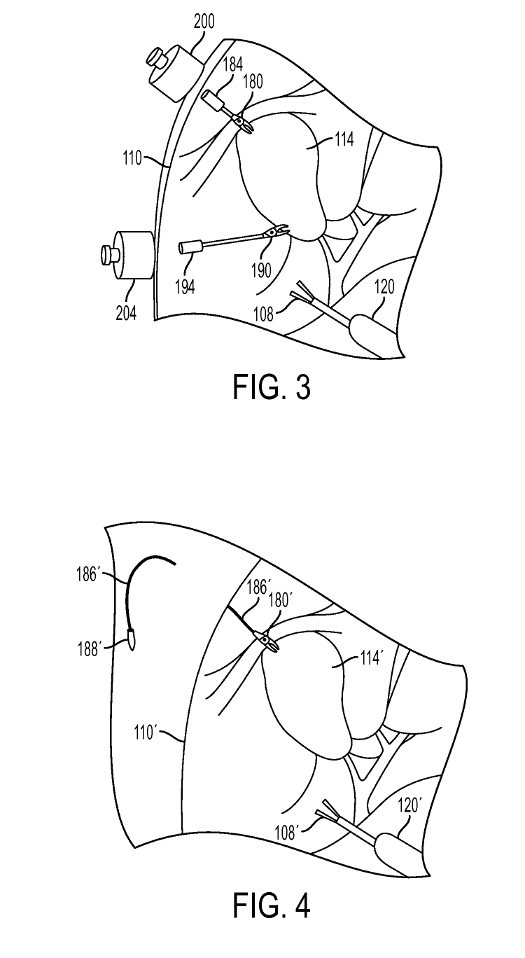

FIG. 3 illustrates one embodiment of a procedure using multiple end effectors that can be performed in view of the disclosed methods and devices. As shown, the end effectors are first and second clamps 180, 190 that are disposed through an abdominal wall 110 and inside an abdominal cavity, where the first and second clamps 180, 190 are then operated to grasp tissue 114 within the cavity to assist in moving and manipulating the tissue 114. The clamps 180, 190, which can be traumatic or atraumatic, can be introduced to the surgical site in a manner as described herein, or using other methods known in the art, and in one exemplary embodiment they include a magnetic portion 184, 194. The magnetic portions 184, 194 can be used in conjunction with external control units 200, 204 to assist in moving the clamps 180, 190 within the cavity to desired locations.

As shown, the external control units 200, 204 can be disposed outside of the body and can be operated to move the clamps 180, 190, and thus the tissue 114 attached thereto, in any desired direction. Optionally, additional control can be provided by an end effector 108 attached to an instrument 120. The instrument 120 can be used to move and manipulate the end effector 108 to engage, move, and manipulate the tissue 114 in manners known in the art. Using multiple end effectors simultaneously at a surgical site can allow a surgeon to more precisely control the movement of tissue within the body. For example, using multiple end effectors can allow tissue to be stretched as desired. Once one or more portions of the tissue 114 are moved to desired locations, procedures can be performed to set the location of various portions of the tissue 114, such as by stapling it. In some embodiments, tissue can be moved to and set at one location and subsequently moved to and set at one or more other locations throughout the course of the procedure.

FIG. 4 illustrates another embodiment of a procedure that can be performed in view of the disclosed methods and devices. As shown, a clamp 180′ is disposed through an abdominal wall 110′, inside an abdominal cavity, and is clamped to tissue 114′ using methods described herein and/or known in the art. The clamp 180′ can be traumatic or atraumatic and can optionally have magnetic properties to assist in control and movement thereof. As shown, a suture 186′ is attached to a proximal end 180p′ of the clamp 180′ and has a needle 188′ attached thereto at its other end. The needle 188′ can be selectively inserted into and out of the body through the abdominal wall 110′ to both manipulate a location of the tissue 114′ attached to the clamp 180′, due at least in part to the tension in the suture 186′, and to set a location of the tissue 114′ by suturing it to a desired location. Other mechanisms, including multiple end effectors, an instrument shaft, and an external control unit, can also be used in conjunction with the clamp 180′, suture 186′, and needle 188′ to manipulate a location of tissue. In the illustrated embodiment, an instrument 120′ having an end effector 108′ can be used to assist with controlling the movement and manipulation of the tissue 114′.

Any number of procedures requiring tissue movement and/or manipulation can be performed at any number of locations in a subject in view of the methods and devices disclosed herein, and thus such procedures are not limited to the procedures in an abdominal or stomach region as discussed above. Examples of other locations at which the disclosed methods and devices can be used include a pelvic area, thorax area, colon, gall bladder, and esophagus, among others. Some examples of surgical procedures, and devices that can be used in conjunction with the same, for use in accordance with the disclosures herein can be found in International Publication No. WO 2009/019288, entitled “Magnetic Surgical Device to Manipulate Tissue in Laparoscopic Surgeries or Via Natural Holes Performed with a Single Trocar,” published on Feb. 12, 2009, which is hereby incorporated by reference in its entirety.

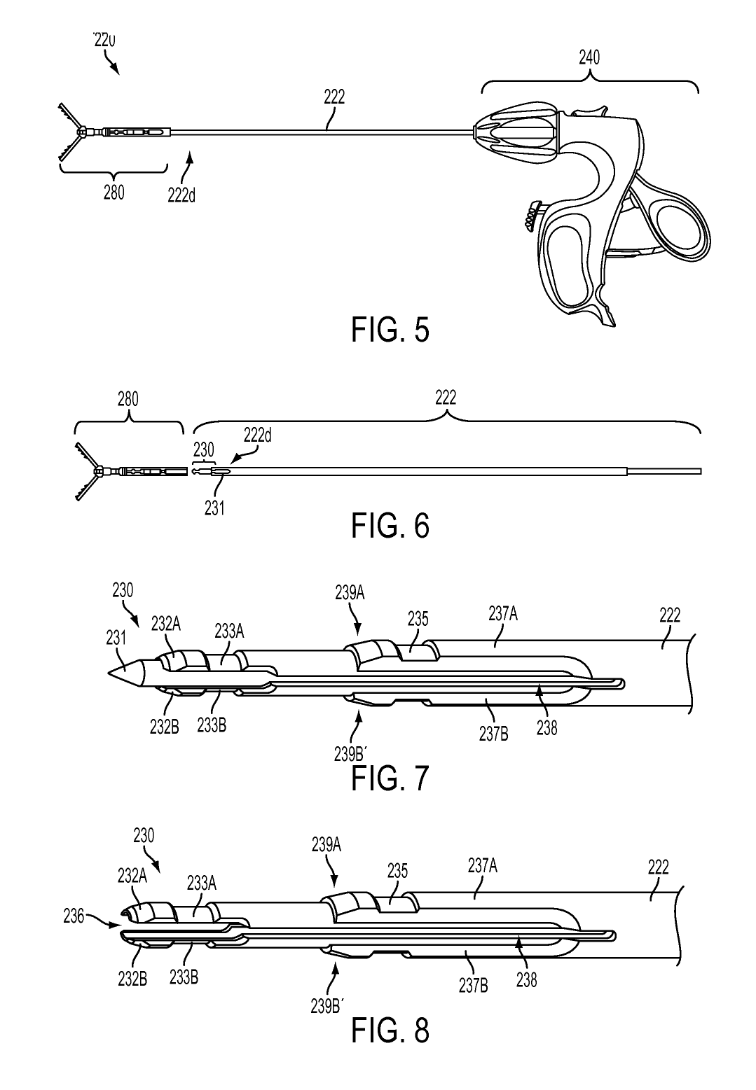

FIGS. 5-15B illustrate one exemplary embodiment of a percutaneous device or instrument 220 for use with detachable end effectors in the described methods. As illustrated in FIG. 5, the device 220 generally includes a shaft 222 for receiving end effectors and a handle 240 for controlling the shaft 222 and/or end effectors associated therewith, which as shown is a tissue grasper 280. An attachment mechanism 230, shown in FIGS. 6-8, can be located at a distal end 222d of the shaft 222 and can be configured to removably and replaceably mate with a variety of end effectors.

FIGS. 7 and 8 illustrate a detailed view of one embodiment of an attachment mechanism 230 located at the distal end 222d of the shaft 222. The attachment mechanism 230 includes a mating feature, which as shown is a circumferential groove 235 located on a lateral surface of shaft arms 237A, 237B. The shaft arms 237A, 237B can be resiliently flexible, i.e., flexibly-tensioned, into an opening 238. The attachment mechanism 230 can also include second arms 232A, 232B projecting distally from distal ends 239A, 239B of the shaft arms 237A, 237B. The second arms 232A, 232B can be axially slidable relative to the shaft 222 and resiliently deflectable, i.e., flexibly-tensioned, medially into a gap 236. The second arms 232A, 232B can include one or more mating features, which as shown include a stepped lateral notch 233A, 233B. An elongate pin or obturator 231 can be positioned medially relative the second arms 232A, 232B and the shaft arms 237A, 237B and can be axially slidable relative to the arms between a locked position in which medial deflection of the arms is prevented (as shown in FIG. 7) and an unlocked position in which medial deflection of the arms is permitted (as shown in FIG. 8). The obturator 231 and second arms 232A, 232B can each slide independently relative to the shaft 222 and the shaft arms 237A, 237B.

As shown in the embodiment of FIG. 7, the obturator 231 can include a pointed distal tip. In this configuration the obturator 231 can be used to form punctures in tissue. Distal ends of the second arms 232A, 232B and distal ends 239A, 239B of the shaft arms 237A, 237B can include tapered surfaces to facilitate passing through an incision and/or mating with an end effector.

A person skilled in the art will recognize that the shaft 222 and its attachment mechanism 230 can have a variety of shapes and sizes and can be made from a variety of materials, depending at least in part on the procedure being performed, the size and type of incisions and ports being used, and the other instruments, devices, and end effectors with which the shaft is being used. Preferred materials for forming the shaft allow the shaft to have a degree of flexibility, and can include a variety of materials known to a person skilled in the art. The shaft is shown as being generally cylindrical, although it can take the form of a number of other shapes. Although sizes of the shaft, such as its diameter and its length, can depend on the other components with which it is used and the procedure in which it is being used, generally a diameter of the shaft can be in the range of about 1 millimeter to about 10 millimeters, and in one embodiment the diameter is about 3 millimeters, and generally a length of the shaft can be in the range of about 50 millimeters to about 300 millimeters, more specifically in the range of about 200 millimeters to about 300 millimeters, and in one embodiment the length is about 200 millimeters.

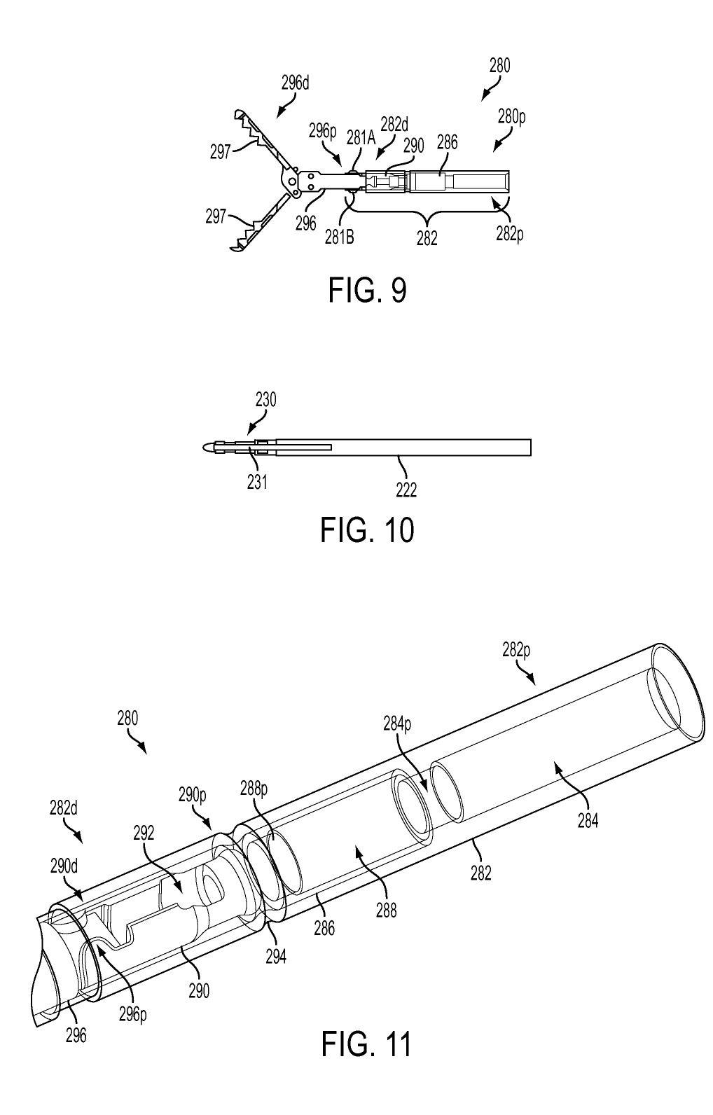

End effectors can be interchangeably attached and detached from the shaft of an instrument, for instance by threadingly or otherwise connecting the shaft and the end effectors. End effectors can include a number of tools that serve a variety of different purposes, including grasping, moving, cutting, and attaching tissue. One exemplary embodiment of an end effector 280 configured for grasping tissue is shown in FIGS. 9 and 11-14B. The end effector 280 can generally includes a receiving tube or sleeve 282 and a tool 296 coupled to a distal end 282d thereof. As shown, the proximal end 280p of the end effector 280 is configured to couple to the shaft 222 of the instrument 220 at the shaft's attachment mechanism 230, thereby allowing the end effector 280 to be operated to engage and/or manipulate tissue at a surgical site by the handle 240 of the instrument 220. In the illustrated embodiment the proximal end 280p includes a receiving sleeve 282 that includes a lock sleeve 286 and a coupler 290 disposed therein.

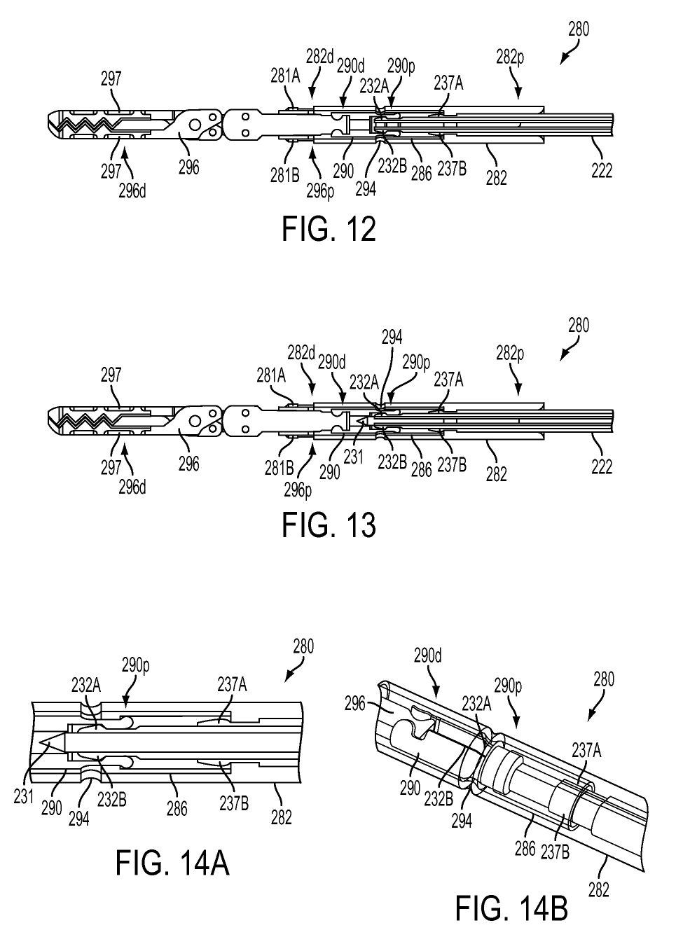

The receiving sleeve 282 is the portion of the end effector 280 that is configured to mate the end effector 280 to the attachment mechanism 230. The receiving sleeve 282 is elongate and generally cylindrical in shape and includes a bore 284 extending therethrough. The bore 284 can be configured to receive the attachment mechanism 230 and it can include, disposed therein, components configured to assist in releasably locking and unlocking the end effector 280 from the shaft 222 of the instrument 220, such as the lock sleeve 286 and coupler 290. The bore 284 can include regions of different diameters that are configured to be complementary to the components to be received by the sleeve 282, such as portions of the attachment mechanism 230. For example, a diameter of the proximal end 284p of the bore 284 can be large enough to allow the attachment mechanism 230 to be inserted therethrough, and the diameter of the bore 284 at a distance distal of the proximal end 284p can be smaller such that the diameter is complementary to the shape and features of the attachment mechanism 230, e.g., the shaft arms 237A, 237B as shown in FIGS. 12-14B.

Optionally, a distal end 282d of the receiving sleeve 282 can include resilient arms 281A, 281B to receive the tool 296, thus providing additional stability for the tool 296 when it is attached to the instrument 220 by way of the coupler 290. Further, an indentation feature 294, which is disposed between the locking sleeve 286 and the coupler 290, can be included as part of the receiving sleeve 282 to assist in retaining a location of the lock sleeve 286 in the receiving sleeve 282. In one exemplary embodiment this feature 294 is formed after the lock sleeve 286 and coupler 290 are disposed within the receiving sleeve 282.

The lock sleeve 286 is disposed within the receiving sleeve 282 and is configured to receive the attachment mechanism 230, and in particular the distal ends 239A, 239B of the shaft arms 237A, 237B, when the instrument 220 is in the locked position. The shape of the lock sleeve 286 is complementary to the shapes and features of the receiving sleeve 282 and attachment mechanism 230, and thus in the illustrated embodiment is generally cylindrical. A bore 288 for receiving the attachment mechanism 230 extends through the sleeve 286 and can include regions of different diameters that are complementary to the components to be received by sleeve 286. For example, a diameter of the proximal end 288p of the bore 288 can be large enough to allow the attachment mechanism 230 to be inserted therethrough, and the diameter of the bore 288 at a distance distal of the proximal end 288p can be smaller such that the diameter is complementary to the shape and features of the proximal end 290p of the coupler 290, as shown in FIGS. 12-14B.

The coupler 290 is also disposed within the receiving sleeve 282 and is configured to receive both the attachment mechanism 230, in particular the second arms 232A, 232B, and the tool 296 when the instrument 220 is in the locked position. The shape of the coupler 290 is generally complementary to the receiving sleeve 282, the attachment mechanism 230, and a proximal end 296p of the tool 296, and thus in the illustrated embodiment it is generally cylindrical with a bore 292 extending at least partially therethrough. Although the coupler 290 is generally cylindrical, in the illustrated embodiment a portion of its top is exposed, thereby making it easier to substitute tools into and out of the coupler 290. The diameter of the bore 292 can vary multiple times along its length. As shown in FIGS. 12-14B, the diameter of the bore 292 at a proximal-most end of the coupler 290 can be configured to receive a portion of the shaft proximal of the second arms 232A, 232B, the diameter of the bore 292 at a second portion can be configured to receive the second arms 232A, 232B, and the diameter of the bore 292 at a third portion can be configured to receive the pin or obturator 231 disposed through the shaft 222. As also shown, the distal-most portion of the bore 292 can be configured to be complementary to the proximal end 296p of the tool 296, thereby making it easier to exchange and substitute tools into and out of the coupler 290.

The tool or component 296 can be any device or instrument configured for use at a surgical site. As shown, the tool 296 is an alligator-style tissue grasper having opposed jaws 297 at its distal end 296d. The jaws 297 can be used to grasp, hold, and lock tissue to move and/or secure it in a desired location. A proximal end 296p of the tool 296 can be configured to detachably couple to the distal end 290d of the coupler 290. The attachment between the two components can be achieved in any number of manners known to those skilled in the art, including by way of an interference fit, a detent, a bayonet, or a rotational fit.

Other embodiments of tools or components that can be used in conjunction with the devices and methods disclosed herein include other types of tissue grasping components or manipulators, such as a Maryland dissector, bi-polar forceps, a stapler, or ultrasonic shears, or another type of tool entirely, such as a laparoscope or a cleaning tool. Exemplary embodiments of such end effectors are described in further detail in previously mentioned U.S. Patent Application Publication No. 2011/0087265, entitled “Laparoscopic Instrument with Attachable End Effector,” filed Oct. 9, 2009, U.S. Patent Application Publication No. 2011/0087266, entitled “Loader for Exchanging End Effectors In Vivo,” filed Oct. 9, 2009, U.S. Patent Application Publication No. 2011/0087267, entitled “Method for Exchanging End Effectors In Vivo,” filed Oct. 9, 2009, U.S. Patent Application Publication No. 2010/0063538, entitled “Surgical Grasping Device,” filed Sep. 9, 2008, U.S. patent application Ser. No. 12/873,845, entitled “Improved Minimally Invasive Surgery,” filed Sep. 1, 2010, U.S. Pat. No. 5,441,059, entitled “Method of Minimally Invasive Surgery,” issued on Aug. 15, 1995, and U.S. Pat. No. 5,352,219, entitled “Modular Tools for Laparoscopic Surgery,” issued on Oct. 4, 1994, as well as U.S. patent application Ser. No. ______, entitled “Devices and Methods for Providing Suction and/or Irrigation in a Surgical Procedure,” filed on even date herewith [Attorney Docket No. 100873-535 (END6966USNP)], which is hereby incorporated by reference in its entirety.

A person skilled in the art will understand that the shapes and sizes of each of the receiving sleeve 282, the lock sleeve 286, the coupler 290, and the tool 296 can change to accommodate various features and sizes of these components, along with the other components with which they are used, such as the attachment mechanism 230 and the obturator 231. In one exemplary embodiment the outer diameter of the receiving sleeve 282 and the tool 296 in a closed configuration that it is suitable for entry through a port is in the range of about 3 millimeters to about 12 millimeters, and more particularly can be about 5 millimeters or about 10 millimeters. Although any number of materials can be used to form each of the receiving sleeve 282, the lock sleeve 286, the coupler 290, and the tool 296, it can be preferable to include a magnetic material in at least a portion of the end effector 280, for instance in a proximal portion 282 of the receiving sleeve 282.

By including a magnetic material as part of the end effector 280, the end effector 280 can be moved in the body independent of the instrument 220 or another shaft. In one exemplary embodiment, illustrated in FIGS. 2C and 2D, the end effector, illustrated as the end effector 80′, can be moved and manipulated by an external control unit 100 that includes a magnetic material complementary to the magnetic material located at a proximal portion 80p′ of the end effector 80′ without the need to have an instrument shaft attached thereto. Movement of the external control unit 100 promotes movement of the end effector 80′, and tissue attached thereto. The external control unit 100, and thus the end effector 80′, can be moved in any direction, including laterally, axially, and rotationally. The external control unit 100 can be located remote from the surgical site, for example external of the subject as shown, or it can be disposed more proximate to the surgical site, such as within the body and external of the cavity in which the end effector 80′ is disposed. Although the illustrated embodiment shows an external control unit 100 that is magnetic, an external control unit can be any device configured to manipulate an end effector within a subject, and thus can include, by way of non-limiting examples, a second shaft, separate from the shaft of the instrument used to manipulate the tool of the end effector, or a device configured to manipulate the end effector by radio frequency.

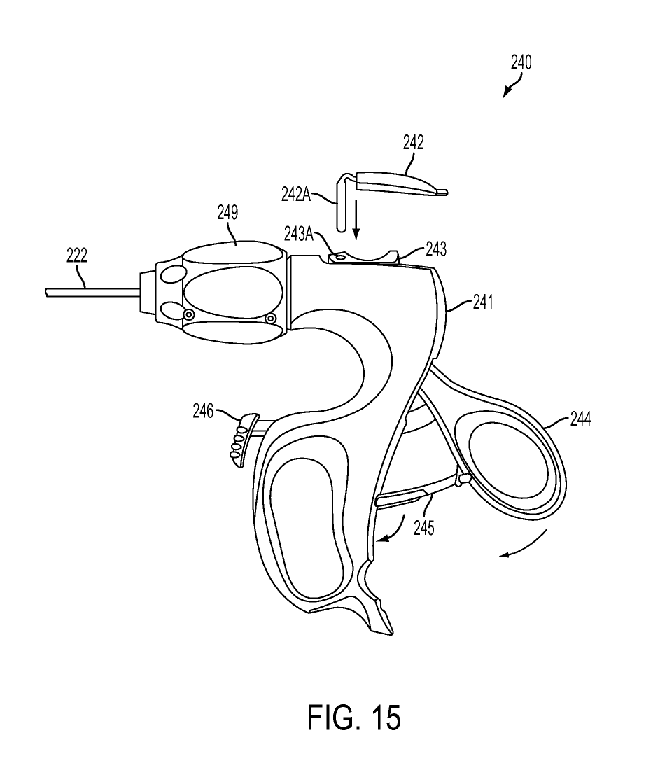

FIG. 15 illustrates an example of the handle 240 of the instrument 220. The handle 240 can include a base 241 and a knob 249. The knob 249 rotates the attachment mechanism 230 disposed at the distal end 222d of the shaft 222 about an axis of the shaft 222, which in turn rotates an attached end effector, illustrated as the end effector 280. The trigger 244 pivots relative to the base 241, causing axial movement of the second arms 232A, 232B and the obturator 231 relative to the shaft 222. In one embodiment, operation of the trigger 244 opens and closes the jaws 297 on an attached end effector 280. The latch 245 pivots relative to the base 241 between a locked position (as shown in FIG. 15) to prevent operation of the trigger 244 and an unlocked position recessed in the base 241, thereby allowing operation of the trigger 244. When mating the instrument 220 to the end effector 280, the latch 245 can be locked so that corresponding matting features of the attachment mechanism 240 and the end effector 280 remain stationary, thereby resulting in a single “snap” feedback. The trigger lock 246 can lock/unlock the trigger 244 in/from its depressed position. An actuator 243, which in this embodiment is a slider, controls axial movement of the obturator 231 relative to the second arms 232A, 232B. The distal-most position of the actuator 243 relative to the base 241 (as shown in FIG. 15) places the obturator 231 in its locked position, and the proximal-most position places the obturator 231 in its unlocked position. The obturator lock 242 includes a pin 242A, which when inserted into the hold 243A maintains the obturator 231 and second arms 232A, 232B in the extended and locked positions.

In one exemplary use of the instrument 220, as shown in FIGS. 9 and 11, a tool 296 for manipulating tissue is selected and is coupled to the distal end 290d of the coupler 290 of the end effector 280. A shaft 222 of an instrument 220 having an attachment mechanism 230, as shown in FIG. 10, is also selected. The end effector 280 and the shaft 222 can then be separately inserted into a subject and subsequently coupled together in vivo. For example, the end effector can be inserted through a port and the shaft 222 can be inserted through an incision formed by the obturator 231, or it can be inserted through a port. Alternatively, the end effector 280 can be attached to a loader and inserted through a port, such as one formed in the umbilicus. In this manner the proximal portion 280p of the end effector 280 can be coupled in vivo to the attachment mechanism 230 of the shaft 222, as shown in FIG. 12.

A surgeon can ascertain that the shaft 222 is properly coupled to the end effector 280 by way of a visual inspection tool, such as an endoscope, or a number of different features incorporated into the instrument 220 and/or the end effector 280. For example, the shaft 222 can have a threshold line marked on it such that once the shaft 222 is properly disposed within the end effector 280, the threshold line is no longer visible. The threshold line can be formed by making the shaft 222 of two different materials, each having a different color, or alternatively, can result from coloring the portion of the shaft on one side of the threshold line different than the color of the portion of the shaft on the other side of the threshold line. Alternatively, the system can be designed such that an audible sound can result once the shaft 222 is fully disposed in the end effector 280, for example as a result of the second arms 232A, 232B moving from a larger diameter to a smaller diameter within the end effector 280 and flexing outwardly upon insertion therein.

Although the embodiment in FIG. 12 shows the shaft 222 coupled to end effector 280, the two components are not locked with respect to each other. The flexibly-tensioned arms 232A, 232B, 237A, 237B are still capable of moving out of alignment with the end effector 280 because the arms can still flex. However, as shown in FIGS. 13-14B, by inserting the obturator 231 through the shaft 222, the obturator 231 radially extends the arms 232A, 232B, 237A, 237B, thereby locking the end effector 280 with respect to the shaft 222. Thus, it is the obturator 231 that can selectively lock or unlock the end effector 280 from the shaft 222.

In one exemplary embodiment the obturator 231 is moved axially by the pin lock 242 disposed on the handle 240. As the pin lock 242 is slid distally and proximally, so too is the obturator 231. A surgeon can ascertain that the obturator 231 is fully disposed in and locked to the shaft 222 by at least one of an audio and visual cue. An audio cue can include the pin lock 242 popping upward and clicking when it reaches its distal-most position. A visual cue can be a color that displays on or near the pin lock 242 when it reaches its distal-most position. For example, a green color located on a side of the pin lock 242 can be exposed when the pin lock 242 pops upward upon reaching its distal-most position. When the instrument 220 is operating the end effector 280, the obturator 231 should generally be in the locked position.

Once the end effector 280 is locked onto the shaft 222, the surgeon can use the handle 240 to manipulate the tissue. For instance, the trigger 244 can be used to open and close the jaws 297 of the tool 296 to grasp and hold tissue. Further, the tool 296 can be rotated by rotating the knob 249. Once the desired tissue movements and manipulations have been performed, the end effector 280 can be disassociated from the instrument 220. Thus, in one exemplary embodiment, the jaws 297 can grasp and hold tissue, and then while still holding the tissue within the jaws 297, the obturator 231 can be moved proximally by sliding the pin lock 242 proximally to unlock the end effector 280 from the instrument shaft 222. The instrument shaft 222 can be removed from the surgical site with the end effector 280 left in place. Accordingly, the end effector 280 can subsequently be moved, for instance by using the methods described above with respect to FIGS. 2A-2D.

To remove the end effector 280 from the surgical site, a loader can be introduced through the port and one or more external control units can be used to direct the end effector 280 toward the loader so the two can be coupled together. The loader can then be used to remove the end effector 280 from the surgical site. Alternatively, an instrument having a shaft can be inserted into the surgical site and one or more external control units can direct the end effector 280 toward the shaft so the two can be coupled together. However, because the diameter of the end effector 280 is typically larger than the diameter of an instrument shaft, removing the end effector 280 by way of the instrument can require the formation of an incision large enough to remove the end effector 280.

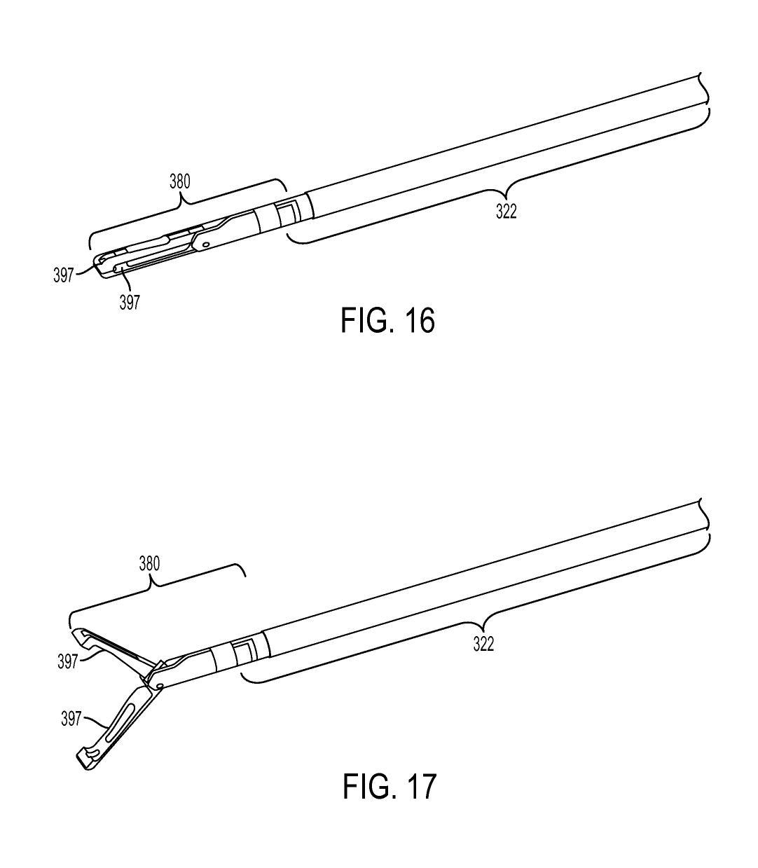

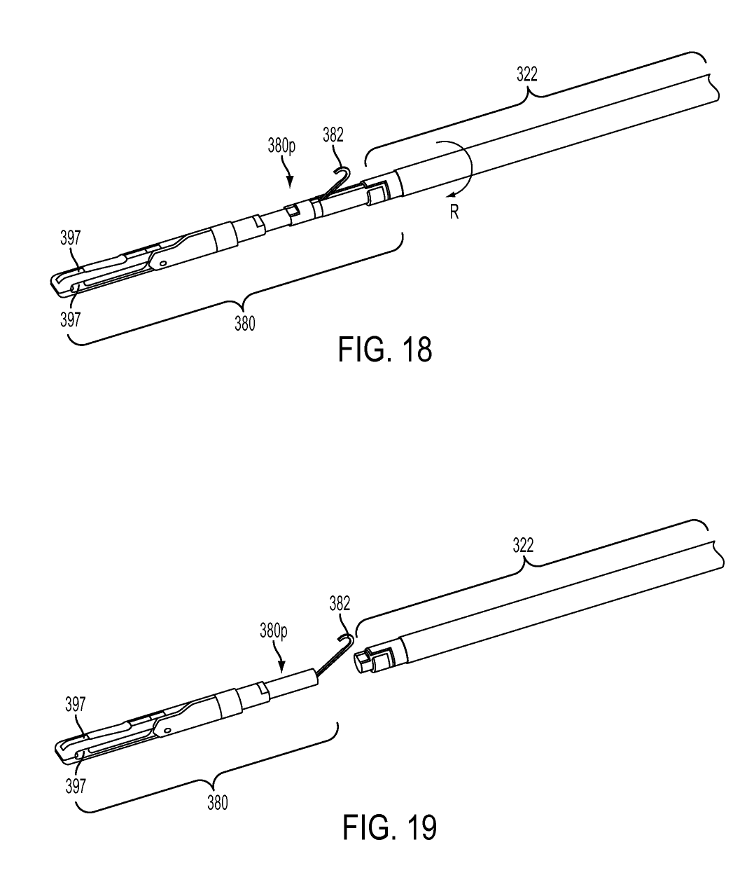

FIGS. 16-19 illustrate an alternative embodiment of an end effector 380 in which the end effector 380 includes a mechanism for securing the end effector 380 at a desired location, as illustrated one or more deployable hooks 382. Such hooks can be useful to allow a surgeon to maintain a location of one or more end effectors at a surgical site without having to re-dock the end effectors to shafts or operate an external control unit to maintain the locations of the end effectors. This also frees up additional space at the surgical site, making it easier to move and manipulate tissue.

As shown, the end effector 380 is generally coupled to a shaft 322 of an instrument and can include jaws 397 that can be opened (FIG. 17) and closed (FIGS. 16 and 18) to grasp and lock tissue as known in the art and described herein. As illustrated in FIG. 18, one or more selectively deployable hooks 382 can be disposed at a proximal end 380p of the end effector 380. The hooks 382 can be exposed in a number of ways, but in one embodiment the instrument includes a central plunger disposed in the shaft 322 and configured to expose the hooks 382 when the plunger is moved distally. Subsequently, the end effector 380 can be disconnected from the shaft 322, for instance by rotating the shaft 322 in a direction R as shown, or by using other methods either known in the art or described herein. Disconnecting the end effector 380 from the shaft 322 leaves the end effector 380 with deployable hooks 382 at the surgical site, free of any instrument shaft. The central plunger can also be used to recover the hooks, for example by moving the plunger proximally to retract the hooks, and in some embodiments can help actuate the end effector and/or be used to help reattach the end effector to the shaft after the end effector has been detached from the shaft.

The exposed hooks 382 can be used to engage tissue, such as an abdominal wall, to hold or hang the end effector 382 at a desired location. A person skilled in the art will recognize that the hooks 382 can engage tissue in a number of different ways. In one exemplary embodiment a separate grasping tool can be introduced to the surgical site and can be used to grasp one of the hooks 382 or the tissue to engage one with the other. In two other alternative embodiments, the hooks 382 can be configured to deploy or shoot outward to engage adjacent tissue when the plunger is moved distally to expose the hooks, or, prior to disconnecting the end effector 380 from the shaft 322, the instrument including the shaft 322 used to first operate the end effector 380 can be used to manipulate and move the end effector 380 to engage the exposed hooks into adjacent tissue. The end effector 380 can then be maintained at the hung location for a period of time, for instance while additional end effectors or instruments are introduced to the surgical site, or until an external control unit is operated to manipulate and move the end effector.

Additional end effectors can be introduced to the surgical site. A benefit of using end effectors that include a mechanism for securing end effectors at a desired location such as deployable hooks is that each of the end effectors disposed at the surgical site within the body cavity need not have a shaft attached thereto. The end effectors can then be manipulated using methods described herein, such as using an external control unit. When multiple end effectors are at the surgical site, the end effectors can be operated to spread tissue, as described with respect to FIGS. 3 and 4. Even when only a single end effector is used, the end effector can utilize the hooks to selectively attach and detach from tissue multiple times at multiple locations, for instance to increase tension in tissue as it is being dissected.

A person skilled in the art will recognize that any number, size, and shape of hooks can be incorporated into the end effectors, and the hooks can be configured to engage a variety of different tissue at various locations in the body. In one exemplary embodiment a length of a hook is approximately in the range of about 1 millimeter to about 10 millimeters, and more particularly is about 3 millimeters. Further, although in the illustrated embodiment the mechanism for securing end effectors to a desired location includes selectively deployable hooks, a person skilled in the art will recognize other mechanisms that can secure end effectors to a desired location can also be incorporated into end effectors without departing from the spirit of the invention.

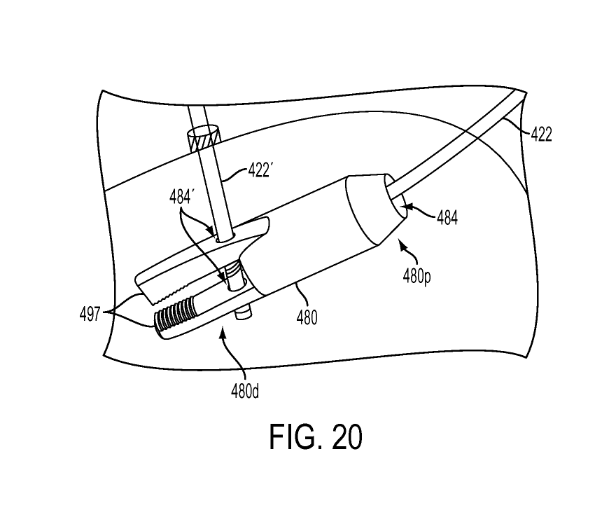

FIG. 20 illustrates an alternative embodiment of an end effector 480 that is configured to receive a second shaft 422′ at its distal end 480d. As shown, the end effector 480 includes a proximal end 480p configured to receive a shaft 422 of an instrument, a distal end 480d having opposed jaws 497 configured to grasp tissue, and a transverse bore 484′ disposed through at least part of each of the jaws 497 for receiving a second shaft 422′ from a location separate from the shaft 422 of the instrument. As shown, the bore 484′ is substantially transverse or perpendicular with respect to a bore 484 in the proximal end 480p that is configured to receive the shaft 422.

The second shaft 422′ can be introduced to the surgical site through its own incision or port that is separate from the ports used to introduce the end effector 480 and shaft 422, or alternatively, it can be introduced through the same port through which the end effector 480 was introduced to the surgical site in instances in which the end effector 480 is coupled to the first shaft 422 in vivo. As shown, the second shaft 422′ can be threadingly mated to the bore 484′ such that the shaft 422′ can be used to manipulate the end effector 480 in a number of ways, including by turning the second shaft 422′ to move it toward the end effector 480 to cause the jaws 497 to move toward each other and tighten, and to move and manipulate tissue engaged by the jaws 497 to another location by serving as an external control unit. Use of the second shaft 422′ as an external control unit can be in lieu of or in addition to using other external control units, such as the magnetic control unit 100 of FIGS. 2C and 2D.

Any of the components and devices known in the art and/or described herein can be provided as part of a kit including an elongate shaft and a plurality of end effectors each configured to removably and replaceably mate to a distal end of the surgical instrument's elongate shaft, as discussed above. The kit can include one or more percutaneous instruments having a handle and a shaft, one or more end effectors, and one or more obturators to selectively lock and unlock end effector(s) from the instrument(s). The end effectors provided in the kit can perform different functions, including but not limited to the functions described herein, and/or can be included together in a single kit to perform a particular function, such as a kit specifically tailored for stretching and stapling tissue. The end effectors can selectively include deployable hooks (as shown in FIGS. 16-19), transverse bores in a distal end thereof for engaging a shaft separate from a shaft of an initial controlling instrument (as shown in FIG. 20), and one or more external control units as described herein. Further, one or more trocars, ports, loaders, and viewing instruments, such as endoscopes or cameras, can be provided to assist in introducing the instruments and end effectors to the surgical site.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the invention described herein will be processed before surgery. First, a new or used instrument is obtained and if necessary cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that device is sterilized. This can be done by any number of ways known to those skilled in the art including beta or gamma radiation, ethylene oxide, steam, and a liquid bath (e.g., cold soak).

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A method for manipulating body tissue, comprising:
grasping tissue disposed within a body cavity at a first location with an end effector that is removably and replaceably attached to a first shaft that extends through a patient's tissue into the body cavity;
detaching the end effector from the first shaft within the body cavity; and
manipulating the end effector following detachment of the first shaft with the grasped tissue secured thereto such that the grasped tissue is moved to a second location within the body cavity.

2. The method of claim 1, further comprising locking the end effector to the grasped tissue.

3. The method of claim 1, wherein, prior to the grasping step, the first shaft is inserted through a first port, and the end effector is inserted through a second port.

4. The method of claim 1, wherein the manipulating step is effected using an external control unit disposed outside of the body cavity.

5. The method of claim 4, wherein the external control unit comprises a magnetic material that is complementary to a material of the end effector.

6. The method of claim 1, wherein the manipulating step is effected by inserting a second shaft into the body cavity, coupling the second shaft to the end effector at a location that is different from the point of coupling of the first shaft to the end effector, and manipulating the second shaft to move the grasped tissue to the second location.

7. The method of claim 6, wherein the first shaft is inserted through a first port, the end effector is inserted through a second port, and the second shaft is inserted through a third port.

8. The method of claim 6, wherein the first shaft is inserted through a first port, the end effector is inserted through a second port, and the second shaft is also inserted through the second port.

9. The method of claim 6, wherein inserting the second shaft occurs while the first shaft is coupled to the end effector.

10. The method of claim 6, wherein the first shaft is mated within a first bore of the end effector and the second shaft is mated within a second bore of the end effector that is substantially perpendicular to the first bore.

11. The method of claim 1, further comprising attaching the end effector with the grasped tissue to other tissue at the second location to maintain the end effector at the second location.

12. The method of claim 11, wherein attaching the end effector with the grasped tissue to other tissue comprises hooking the end effector to the other tissue using a hook of the end effector.

13. A device for manipulating tissue, comprising:
an end effector having a proximal end and a distal end;
a tissue grasping component formed at the distal end of the end effector and configured to engage a first tissue; and
one or more selectively deployable hooks disposed within the end effector and configured to selectively engage additional tissue to maintain the end effector at a desired location,
wherein the end effector is configured to be selectively moved from a first location to a second location by a movement control component.

14. The device of claim 13, further comprising a first bore disposed in the proximal end of the end effector, the first bore extending along a longitudinal axis of the end effector and being configured to mate with a shaft that is effective to operate the end effector to selectively grasp and release the first tissue.

15. The device of claim 14, wherein the tissue grasping component comprises a pair of movable opposed jaws and a second bore extending through at least part of each of the jaws, the second bore being oriented substantially perpendicular to the longitudinal axis of the end effector.

16. The device of claim 15, wherein the second bore and a second shaft mate by way of a threaded connection.

17. A kit for manipulating tissue, comprising:
a threaded shaft having a tissue penetrating distal tip and being configured to be inserted percutaneously through tissue;
an end effector configured to selectively grasp tissue, the end effector having a first bore at a proximal end of the end effector that is oriented along a longitudinal axis of the end effector and a second bore that is distal to the first bore and oriented substantially perpendicular to the first bore, the end effector being configured to be inserted into a body cavity independent of the threaded shaft and to be selectively attached to and detached from the shaft in vivo;
an external control unit configured to move the detachable end effector from a first location to a second location.

18. The kit of claim 17, further comprising a trocar element.

19. The kit of claim 17, wherein the end effector has one or more selectively deployable hooks disposed therein.
